(19) United States
(12) Patent Application Publication
(10) Pub. No.: US 2021/0196146 A1
(43) Pub. Date: Jul. 1, 2021

(54) MEDICAL INSTRUMENT WITH SENSOR FOR USE IN A SYSTEM AND METHOD FOR ELECTROMAGNETIC NAVIGATION

(71) Applicant: Covidien LP, Mansfield, MA (US)

(72) Inventors: Benjamin Greenburg, Hod Hasharon (IL); Alex A. Peterson, Maple Grove, MN (US); David J. Serdar, Shorewood, MN (US); David M. Costello, Delano, MN (US)

(21) Appl. No.: 17/200,998

(22) Filed: Mar. 15, 2021

Related U.S. Application Data
(63) Continuation of application No. 14/919,950, filed on Oct. 22, 2015.
(60) Provisional application No. 62/095,563, filed on Dec. 22, 2014.

(51) Int. Cl. A61B 5/06; A61B 5/00

(52) U.S. Cl. CPC A61B 5/062; A61B 2562/166; A61B 5/6852

(57) ABSTRACT

A medical instrument includes a sensor, a surface, at least one non-conductive material, and at least one pair of contacts. The sensor has at least one coil formed on a conductive material. The surface is suitable for receiving the sensor and can be placed in an EM field. The at least one non-conductive material covers the at least one coil of the sensor. The at least one pair of contacts are electrically connected to the at least one coil and connectable to a measurement device, which senses an induced electrical signal based on a magnetic flux change of the EM field. The location of the medical instrument in a coordinate system of the EM filed is identified based on the induced electrical signal in the sensor.

### CROSS-REFERENCE TO RELATED APPLICATIONS

This application is a continuation of U.S. patent application Ser. No. 14/919,950, filed on Oct. 22, 2015, which claims the benefit of the filing date of provisional U.S. Patent Application No. 62/095,563, filed on Dec. 22, 2014.

### FIELD

The present disclosure relates to a medical instrument including a sensor, and a system in which the location of the sensor can be detected and tracked. More particularly, the present disclosure relates to systems and methods that identify a location of a medical instrument having the sensor in an electromagnetic field.

### BACKGROUND

Electromagnetic navigation (EMN) has helped expand the possibilities of treatment to internal organs and diagnosis of diseases. EMN relies on non-invasive imaging technologies, such as computed tomography (CT) scanning, magnetic resonance imaging (MRI), or fluoroscopic technologies. These images may be registered to a location of a patient within a generated magnetic field, and as a result the location of a sensor placed in that field can be identified with reference to the images. As a result, EMN in combination with these non-invasive imaging technologies is used to identify a location of a target and to help clinicians navigate inside of the patient's body to the target.

In one particular example of currently marketed systems in the area of locating the position of medical instruments in a patient's airway, a sensor is placed at the end of a probe referred to as a locatable guide and passed through an extended working channel (EWC) or catheter, and the combination is inserted into the working channel of a bronchoscope. The EWC and probe with sensor is then navigated to the target within the patient. Once the target is reached, the locatable guide (i.e., sensor and probe) can be removed and one or more instruments, including biopsy needles, biopsy brushes, ablation catheters, and the like can be passed through the working channel and EWC to obtain samples and/or treat the target. At this point, however, because the locatable guide with its sensor have been removed, the exact location of a distal end of the EWC, and by extension any instrument which might be passed there through is not precisely known.

Images generated by the non-invasive imaging technologies described above do not provide the resolution of live video imaging. To achieve live video, a clinician may utilize the features of an endoscope. However, an endoscope is limited by its size and as a result cannot be navigated to the pleura boundaries of the lungs and other very narrow passageways as is possible with tools typically utilized in EMN. An alternative is a visualization instrument that is inserted through the EWC and working channel of the endoscope, which can be sized to reach areas such as the pleura boundaries.

As with the locatable guide, however, once the visualization instrument is removed the location of the distal end of the EWC is unclear. One technique that is used is the placement of one or more markers into the tissue near the target and the use of fluoroscopy to confirm location of the EWC and the markers, and any subsequent instruments passed through the EWC. Due to the small diameter of the EWC, simultaneous insertion of more than one instrument may be impractical. Thus, repeated insertions and removals of instruments for visualization, diagnosis, and surgeries are necessitated. Such repeated insertions and removals lengthen diagnostic or surgical time and efforts, and increase costs on patients correspondingly. Thus, it is desirous to make a fewer insertion and/or removal of instruments to shorten times necessary for diagnosis and surgeries while at the same time increasing the certainty of the location of the EWC and instruments passed through the EWC, including imaging modalities.

### SUMMARY

In an embodiment, the present disclosure features a medical instrument that identifies its location in an electromagnetic (EM) field by a sensor. The medical instrument includes a sensor, a surface, at least one non-conductive material, and at least one pair of contacts. The sensor has at least one coil formed on a conductive material. The surface is suitable for receiving the sensor and can be placed in an EM field. The at least one non-conductive material covers the at least one coil of the sensor. The at least one pair of contacts are electrically connected to the at least one coil and connectable to a measurement device, which senses an induced electrical signal based on a magnetic flux change of the EM field. The location of the medical instrument in a coordinate system of the EM filed is identified based on the induced electrical signal in the sensor.

In an aspect, the conductive material is printed directly on or fabricated separately and attached to a distal portion of the medical instrument. The medical instrument further includes a non-conductive layer on the distal portion of the medical instrument on which the conductive material is printed.

In another aspect, the sensor includes multiple layers of the conductive material and the non-conductive material printed or fabricated on the distal portion of the medical instrument. Each conductive layer has a different configuration, which includes a pitch angle and a number of loops of the conductive material. The conductive layer of each layer of the multiple layers is connected to the conductive layer of another layer through vias.

In yet another aspect, the at least one non-conductive material is fabricated or printed directly on a distal portion of the medical instrument, over the conductive material.

In still another aspect, the sensor is a flex circuit sensor where a conductive layer and a non-conductive layer are formed on a flex substrate, and the flex circuit sensor is attached to the medical instrument. The flex circuit sensor includes a plurality of conductive and non-conductive layers. The conductive layer includes conductive material forming a plurality of coils. The conductive material of each conductive layer is connected to the conductive material of another conductive layer through vias. Each conductive layer includes two or more separate coils, connected to each other through vias. The flex substrate of the flex circuit sensor is polyimide film. Each conductive layer includes two or more separate coils connected to each other by conductive material printed on another layer. One of the two or more separate coils has a rotational orientation different from a rotational orientation of the other of the two or more separate coils.

In still another aspect, the conductive material forms a helical shape, which is counter clockwise or clockwise.

In yet another aspect, the outer surface of the tube is made of ETFE, PTFE, polyimide, or non-conductive polymer.

In yet another aspect, the conductive material is copper, silver, gold, conductive alloys, or conductive polymer.

In yet still another aspect, the medical instrument is an extended working channel, an imaging instrument, a biopsy forceps, a biopsy brush, a biopsy needle, or a microwave ablation probe.

In another embodiment, the present disclosure features an electromagnetic navigation system that identifies its location in an EM field by a sensor. The EM navigation system includes an EM board, a medical instrument, and a processor. The EM board generates an EM field. The medical instrument includes a sensor, a surface, at least one non-conductive material, and at least one pair of contacts. The sensor has at least one coil formed on a conductive material. The surface is suitable for receiving the sensor and can be placed in an EM field. The at least one non-conductive material covers the at least one coil of the sensor. The at least one pair of contacts are electrically connected to the at least one coil and connectable to a measurement device, which senses an induced electrical signal based on a magnetic flux change of the EM field. The location of the medical instrument in a coordinate system of the EM filed is identified based on the induced electrical signal in the sensor. The processor processes the induced electrical signal to identify a location of the medical instrument in a coordinate system of the EM field.

Any of the above aspects and embodiments of the present disclosure may be combined without departing from the scope of the present disclosure.

### BRIEF DESCRIPTION OF THE DRAWINGS

Objects and features of the presently disclosed systems and methods will become apparent to those of ordinary skill in the art when descriptions of various embodiments are read with reference to the accompanying drawings, of which:

FIG. 1 is a perspective view of a system for identifying a location of a medical instrument in accordance with an embodiment of the present disclosure;

FIG. 2A is a profile view of a catheter guide assembly and medical instrument in accordance with an embodiment of the present disclosure;

FIG. 2B is an enlarged view of the indicated area of detail of FIG. 2A;

FIG. 3A depicts a sensor as a coil wound or printed at the distal portion of a medical instrument in accordance with an embodiment of the present disclosure;

FIGS. 3B-3E are perspective views of a plurality of medical instruments in accordance with an embodiment of the present disclosure;

FIG. 4A is a sensor in a form of a flex circuit in accordance with an embodiment of the present disclosure;

FIG. 4B is an expanded view of a distal portion of a medical instrument around which the flex circuit of FIG. 4A wraps in accordance with an embodiment of the present disclosure;

FIG. 5 is an illustrative design of a sensor including two-coils in a multi-layer flex circuit in accordance with an embodiment of the present disclosure;

FIG. 6 is an illustrative design of two sensor in a multi-layer flex circuit in accordance with an embodiment of the present disclosure;

FIG. 7 is an illustration of a printer that prints a sensor on a surface of a medical instrument in accordance with an embodiment of the present disclosure; and

FIG. 8 is a flowchart of a method for printing a sensor on a medical instrument in accordance with an embodiment of the present disclosure.

### DETAILED DESCRIPTION

The present disclosure is related to medical instruments, systems and methods for identifying a location of medical instruments in an electromagnetic field by using a sensor. The sensors may be fabricated directly on or separately fabricated and then affixed to the medical instruments, including imaging instruments. One method of fabricating the sensors is via printing. Since the sensor may be inserted inside of patient's body with medical instruments, the location of the medical instruments is identified real-time. Further, the sensor may provide and trace an exact direction and location of the medical instrument with other imaging modality. Due to the small size of the sensor, medical instruments may incorporate the sensor inside or outside of the medical instruments, to facilitate continuous navigation. Although the present disclosure will be described in terms of specific illustrative embodiments, it will be readily apparent to those skilled in this art that various modifications, rearrangements, and substitutions may be made without departing from the spirit of the present disclosure. The scope of the present disclosure is defined by the claims appended to this disclosure.

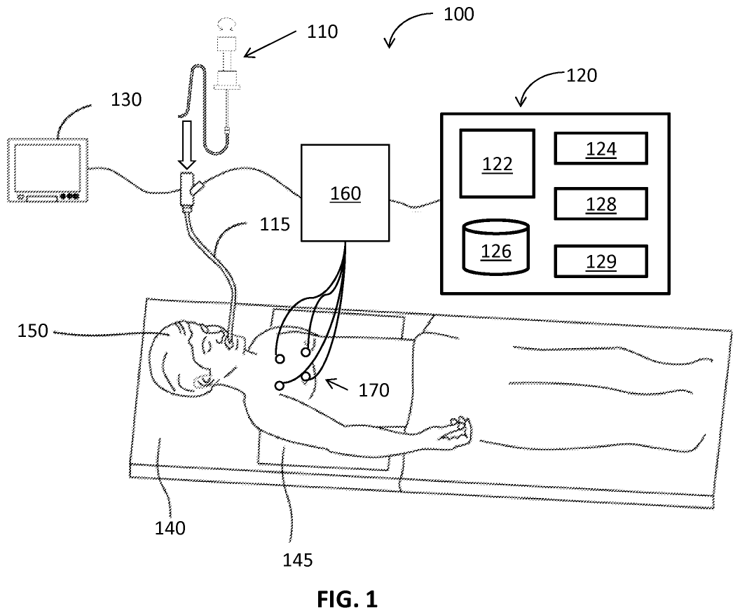

FIG. 1 illustrates one illustrative embodiment of a system and method for identifying a location of medical instruments in an electromagnetic field. In particular, an electromagnetic navigation (EMN) system 100, which is configured to utilize CT, MRI, or fluoroscopic images, is shown. One such EMN system may be the ELECTROMAGNETIC NAVIGATION BRONCHOSCOPY® system currently sold by Covidien LP. The EMN system 100 includes a catheter guide assembly 110, a bronchoscope 115, a computing device 120, a monitoring device 130, an EM board 140, a tracking device 160, and reference sensors 170. The bronchoscope 115 is operatively coupled to the computing device 120 and the monitoring device 130 via a wired connection (as shown in FIG. 1) or wireless connection (not shown).

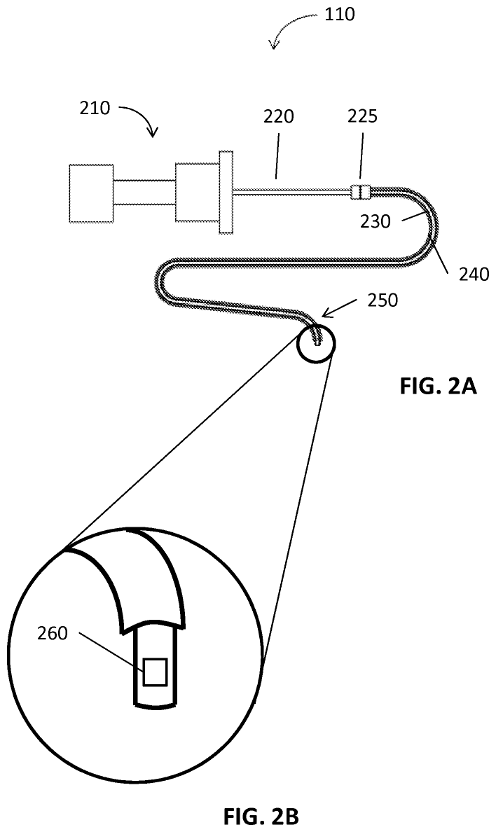

FIG. 2A illustrates an embodiment of the catheter guide assembly 110 of FIG. 1. The catheter guide assembly 110 includes a control handle 210, which enables advancement and steering of the distal end 250 of the catheter guide assembly 110. The catheter guide assembly 110 includes a locatable guide catheter (LG) 220 inserted in the EWC 230 and an electromagnetic EM sensor 260, as shown in FIG. 2B. A locking mechanism 225 secures the EWC 230 and the LG 220 to one another. Catheter guide assemblies usable with the instant disclosure may be currently marketed and sold by Covidien LP under the name SUPERDIMENSION® Procedure Kits and EDG™ Procedure Kits. For a more detailed description of the catheter guide assemblies, reference is made to commonly-owned U.S. patent application Ser. No. 13/836,203 filed on Mar. 15, 2013, by Ladtkow et al. and U.S. Pat. No. 7,233,820, the entire contents of which are incorporated in this disclosure by reference. As will be described in greater detail below, the EM sensor 260 on the distal portion of the LG 220 senses the electromagnetic field, and is used to identify the location of the LG 220 in the electromagnetic field.

In use, the bronchoscope 115 is inserted into the mouth or through an incision of a patient 150 to capture images of the internal organ. In the EMN system 100, inserted into the bronchoscope 115 is a catheter guide assembly 110 for achieving an access to the internal organ of the patient 150. The catheter guide assembly 110 may include an extended working channel (EWC) 230 into which a locatable guide catheter (LG) 220 with the EM sensor 260 at the distal portion is inserted. The EWC 230, the LG 220, and the EM sensor 260 are used to navigate through the internal organ as described in greater detail below.

In an alternative embodiment, instead of a bronchoscope 115 inserted via a natural orifice the catheter guide assembly 110 is inserted into the patient 150 via an incision. The catheter guide assembly 110 including the extended working channel 230 may be inserted through the incision to navigate a luminal network other than the airways of a lung, such as the cardiac luminal network.

The computing device 120, such as, a laptop, desktop, tablet, or other similar computing device, includes a display 122, one or more processors 124, memory 126, a network card 128, and an input device 129. The EMN system 100 may also include multiple computing devices, wherein the separate computing devices are employed for planning, treatment, visualization, and other aspects of assisting clinicians in a manner suitable for medical operations. The display 122 may be touch-sensitive and/or voice-activated, enabling the display 122 to serve as both input and output devices. The display 122 may display two dimensional (2D) images or a three dimensional (3D) model of an internal organ, such as the lung, prostate, kidney, colon, liver, etc., to locate and identify a portion of the internal organ that displays symptoms of diseases.

The display 122 may further display options to select, add, and remove a target to be treated and settable items for the visualization of the internal organ. In an aspect, the display 122 may also display the location of the catheter guide assembly 110 in the electromagnetic field based on the 2D images or 3D model of the internal organ.

The one or more processors 124 execute computer-executable instructions. The processors 124 may perform image-processing functions so that the 3D model of the internal organ can be displayed on the display 122. In embodiments, the computing device 120 may further include a separate graphic accelerator (not shown) that performs only the image-processing functions so that the one or more processors 124 may be available for other programs. The memory 126 stores data and programs. For example, data may be image data for the 3D model or any other related data such as patients' medical records, prescriptions and/or history of the patient's diseases.

One type of programs stored in the memory 126 is a 3D model and pathway planning software module (planning software). An example of the 3D model generation and pathway planning software may be the ILOGIC® planning suite currently sold by Covidien LP. When image data of a patient, which is typically in digital imaging and communications in medicine (DICOM) format, from for example a CT image data set (or an image data set by other imaging modality) is imported into the planning software, a 3D model of the internal organ is generated. In an aspect, imaging may be done by CT imaging, magnetic resonance imaging (MRI), functional MM, X-ray, and/or any other imaging modalities. To generate the 3D model, the planning software employs segmentation, surface rendering, and/or volume rendering. The planning software then allows for the 3D model to be sliced or manipulated into a number of different views including axial, coronal, and sagittal views that are commonly used to review the original image data. These different views allow the user to review all of the image data and identify potential targets in the images.

Once a target is identified, the software enters into a pathway planning module. The pathway planning module develops a pathway plan to achieve access to the targets and the pathway plan pin-points the location and identifies the coordinates of the target such that they can be arrived at using the EMN system 100, and particularly the catheter guide assembly 110 together with the EWC 230, the LG 220, and the EM sensor 260. The pathway planning module guides a clinician through a series of steps to develop a pathway plan for export and later use during navigation to the target in the patient 150. The term, clinician, may include doctor, surgeon, nurse, medical assistant, or any user of the pathway planning module involved in planning, performing, monitoring and/or supervising a medical procedure.

Details of these processes and the pathway planning module can be found in U.S. patent application Ser. No. 13/838,805 filed by Covidien LP on Jun. 21, 2013, and entitled “Pathway Planning System and Method,” the entire contents of which are incorporated in this disclosure by reference. Such pathway planning modules permit clinicians to view individual slices of the CT image data set and to identify one or more targets. These targets may be, for example, lesions or the location of a nerve which affects the actions of tissue where the disease has rendered the internal organ's function compromised.

The memory 126 may store navigation and procedure software which interfaces with the EMN system 100 to provide guidance to the clinician and provide a representation of the planned pathway on the 3D model and 2D images derived from the 3D model. An example of such navigation software is the ILOGIC° navigation and procedure suite sold by Covidien LP. In practice, the location of the patient 150 in the EM field generated by the EM field generating device 145 must be registered to the 3D model and the 2D images derived from the 3D model. Such registration may be manual or automatic and is described in detail and commonly assigned U.S. Provisional Patent Application 62/020,240 entitled “System and method for navigating within the lung.”

As shown in FIG. 1, the EM board 140 is configured to provide a flat surface for the patient to lie down and includes an EM field generating device 145. When the patient 150 lies down on the EM board 140, the EM field generating device 145 generates an EM field sufficient to surround a portion of the patient 150. The EM sensor 260 at the end of the LG 220 is used to determine the location of the distal end of the LG 220 and therewith the EWC 230 within the patient. In an aspect, a separate EM sensor may be located at the distal end of the EWC 230 and therewith the exact location of the EWC 230 in the EM field generated by the EM field generating device 145 can be identified within the patient 150.

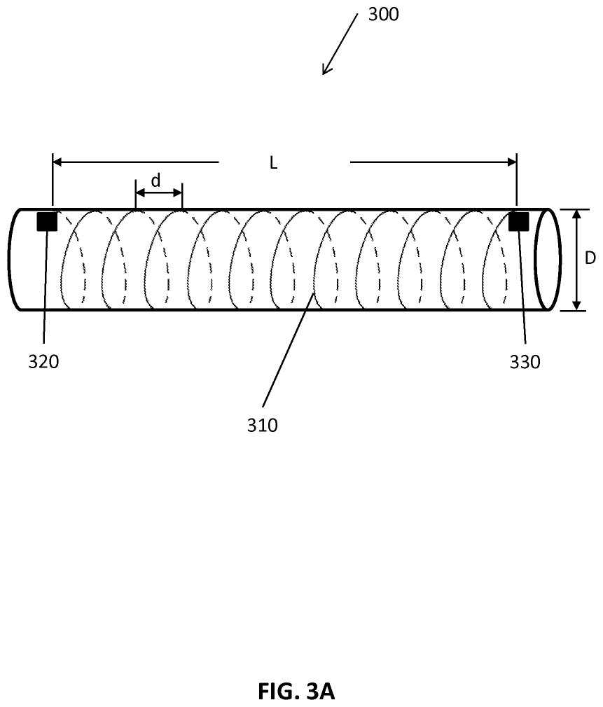

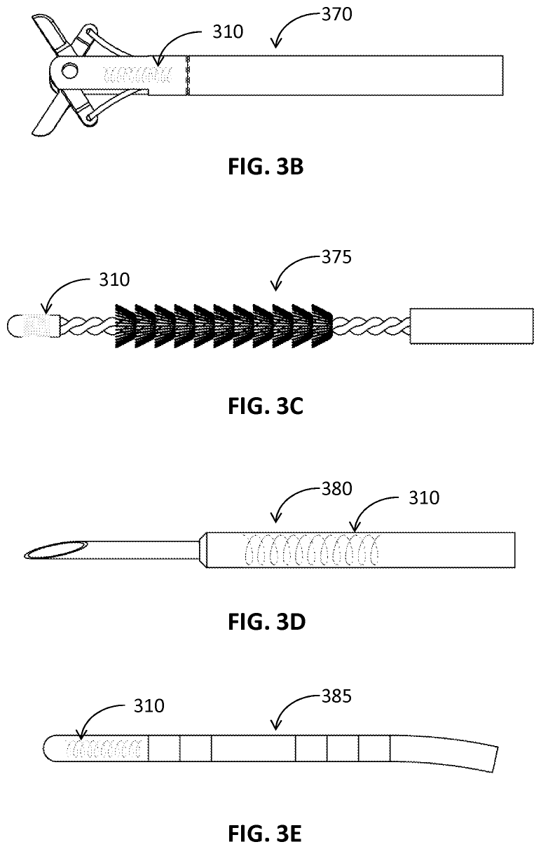

In yet another aspect, the EM board 140 may be configured to be operatively coupled with the reference sensors 170 which are located on the chest of the patient 150. The reference sensors 170 move up following the chest while the patient 150 is inhaling and move down following the chest while the patient 150 is exhaling. The movement of the chest of the patient 150 in the EM field is captured by the reference sensors 170 and transmitted to the tracking device 160 so that the breathing pattern of the patient 150 may be recognized. The tracking device 160 also receives the output of the EM sensor 260, combines both outputs, and compensates the breathing pattern for the location of the EM sensor 260. In this way, the location identified by the EM sensor 260 may be compensated for such that the compensated location of the EM sensor 260 may be synchronized with the 3D model of the internal organ. As noted above, however, the use of an LG 230 with an EM sensor 260 at its distal end 250 can result in challenges surrounding instrument swaps, loss of location information, and a general prolongation of the time needed for a procedure. To alleviate these issues, FIG. 3A depicts an electromagnetic sensor 310 in the shape of a coil. The sensor 310 may be fabricated or printed directly on the distal portion of a medical instrument 300. The fabricated or printed electromagnetic sensor (PES) 310 may form a helical shape, as depicted or in another configuration as required by the application. The instrument 300 may be the EWC 230, a catheter, a biopsy instrument, an ablation instrument, a monopolar or bipolar electrosurgical instrument, an imaging instrument, a marking instrument, or a needle, in short any instrument capable of being inserted into the luminal network (e.g., the airways or vasculature of a patient). In one embodiment the instrument 300 is sized to pass through the EWC 230. Alternatively, the instrument 300 may be the EWC 230. Other exemplary instruments are shown in FIGS. 3B-3E, depicting biopsy forceps 370, a biopsy brush 375, a biopsy needle 380, and a microwave ablation probe 385, each having an EM sensor 310 applied by the methods of the present disclosure.

The distal portion of the instrument 300 may be made of or covered by Ethylene tetrafluoroethylene (ETFE), Polytetrafluoroethylene (PTFE), polyimide, or another suitable material to form a non-conductive base for the sensor 310. If the distal portion of the instrument 300 is not covered or made of a non-conductive material, a non-conductive material must be applied to the distal portion first to form an insulating base for the sensor 310.

With respect to the sensor 310 depicted in FIG. 3A, the coil of sensor 310 is in the shape of a helix. The dimensions of the helix (i.e., the length L, the distance d between two adjacent loops, and a diameter D of the helix, as shown in FIG. 3A) may be chosen to create an optimum sensor 310. A pitch angle a may be used to define the helix and be calculated by:

$$α = {tan}^{- 1} ( \frac{d}{π D} ) .$$

The pitch angle α indicates the density of loops of the fabricated or printed helix along the longitudinal axis of the instrument 300.

In embodiments, the sensor 310 may include multiple layers. Specifically, after a conductive material is applied to the instrument 300 to form a first coil of sensor 310, a non-conductive material may be applied over the first coil, and the second coil formed of a conductive material may be applied over both the non-conductive material and the first coil on the instrument 300. This may continue until a desired number of coils are fabricated or printed on the instrument 300. Each coil may have a different configuration, e.g., a different length L and a different distance d between two adjacent loops of a helix from that of the other coils. Alternatively, each of the multiple coils of the sensor 310 may be applied to different locations of the instrument 300.

In an aspect of the present disclosure, the rotational direction of the helix of one coil may be different from that of another coil. That is, one helix may have the counter clockwise orientation and another one may have the clockwise orientation. In another aspect, the conductive material may be copper, silver, gold, conductive alloys, or conductive polymer, and the non-conductive material may be ETFE, PTFE, non-conductive polymer, or polyimide.

According to a further aspect of the present disclosure, each of the end portions of the helix 310 may have a larger area for electrical contacts 320 and 330 than other areas of conductive material in the helix. Wires are connected to each of the contacts 320 and 330. These wires may extend the length of the catheter assembly 100 and be connected to the tracking device 160. Thus, when the instrument 300 is located within an electromagnetic field, electrical signal (e.g., voltage) may be induced in the sensor 310 while the instrument 300 is moving inside the electromagnetic field. The induced electrical signal is transmitted to the tracking device 160, which calculates a location of the instrument 300 with respect to a coordinate system of the electromagnetic field. This calculated location may be registered to the 3D model so that a computing device may display the location in the 3D model on a display. In this way, the clinician may identify the relative location of the instrument 300 in the 3D model and 2D images of the navigation and procedure software as described above.

The induced voltage is derived from the Maxwell's equations and is calculated by the following equation:

$${ɛ}_{ind} = - N \frac{Δ Φ}{Δ t} ,$$

where ε_{ind} is the induced voltage, N is the number of loops in the helix, ΔΦ is the change of magnetic flux of the electromagnetic field, and Δt is the change in time. The magnetic flux Φ is a product of the magnitude of the magnetic field and an area. In the same way, the change of magnetic flux, ΔΦ, is a product of the change of the magnitude of the magnetic field and the area of the one loop in the helix. Thus, the more loops in the helix, the larger the magnitude of the induced voltage is. And the faster the change of the magnetic flux, the higher the magnitude of the induced voltage is. The negative sign indicates that the induced voltage is created to oppose the change of the magnetic flux.

Since the instrument 300 is typically moved slowly and with some caution inside of the body or in a luminal network of an internal organ and the size of the loops in the helix is to be minimal, the number of loops in the helix may be sufficiently large to compensate the slow movements and the size of the loops in order to have a recognizable induced electrical signal. Thus, when a sensitivity level of the induced electrical signal and a magnitude level of the electromagnetic field are determined, the number of loops in the coil sensor 310 may be determined by the following:

$$N = - \frac{{ɛ}_{ind} Δ t}{ΔΦ} .$$

The sensor 310 may sense different EM fields generated by the EM field generating device 145, in one embodiment employing three coils in the sensor 310 three separate fields are sensed. The strength of the EM field decreases proportionally with the reciprocal of the square of the distance from the source (e.g., the EM field generating device 145). Thus, the magnitude of the voltage induced by an EM field includes information defining the distance of the sensor 310 from the EM field generating device 145. By determining the distance information based on the induced electrical signal, a location of the sensor 310 can be identified with respect to the location of the EM field generating device 145.

In an aspect, where the EM field generating device 145 generates three EM fields, which may have three different directivity patterns such as x-, y-, and z-axes, respectively, induced electrical signal may have different patterns when the instrument 300 having the sensor 310 moves in any direction within the coordinate system of the EM fields. For example, when the instrument 300 moves in the x-axis direction, strengths of EM fields having y- and z-axes directivity patterns will display larger differences as compared to the sensed changes in strength of the EM field having x-axis directivity. Thus, the location of the instrument 300 may be identified by checking patterns of induced voltage sensed by the sensor 310.

In accordance with the present disclosure, sensor 310 may be fabricated or printed directly onto the instrument 300. That is, during the manufacture of the instrument 300, one of the processing steps is to apply one or more conductive inks or other materials to the instrument 300. This printing may be performed by a number of processes including ink jet printing, flexographic printing, vapor deposition, etching, and other known to those of skill in the art without departing from the scope of the present disclosure.

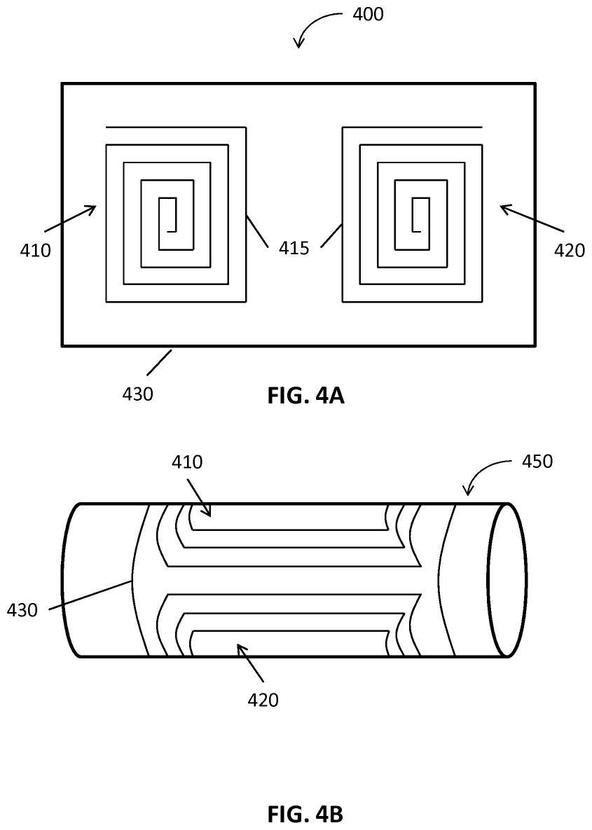

In a further embodiment of the present disclosure, the sensor 310 may be fabricated or printed using one or more of the above-identified techniques to form a flexible circuit which is applied to the instrument 300 using an adhesive or the like. FIG. 4A shows a flex circuit sensor 400 and FIG. 4B shows the flex circuit sensor 400 of FIG. 4A incorporated on a surface of an instrument 450, such as a medical instrument. The flex circuit sensor 400 may have a thickness of about 0.05 millimeter (mm) so that the flex circuit can be applied to, inserted into, or affixed to an instrument without appreciably increasing its dimensions.

In accordance with one embodiment, a conductive material 415 is fabricated or printed on a non-conductive film 430 to form a coil 410 or 420 and a second non-conductive film 430 covers the conductive material. Thus, the coil 410 or 420 is protected by the non-conductive films 430.

The flex circuit sensor 400 may have a first coil 410 and a second coil 420 as shown in FIG. 4A. As described above, in one aspect of the present disclosure, each coil may have a different rotational orientation. The first coil 410 may have the clockwise rotational orientation and the second coil 420 may have the counter clockwise rotational orientation. Nevertheless, when the flex circuit sensor 400 is affixed to or around the instrument 450 so that two coils are facing each other across the longitudinal axis of the tube, the first and second coils 410 and 420 may have the same rotational orientation.

In an aspect, the flex circuit sensor 400 may be affixed to an instrument 450 in a manner such that the flex circuit sensor 400 is bent or made to curve around a portion of the instrument 450. In such a situation, the flex circuit sensor 400 may not be able to sense changes in electromagnetic fields parallel to the flex circuit sensor 400. Thus, in order to accurately sense changes in the electromagnetic fields in multiple directions within an electromagnetic field, the flex circuit sensor 400 including at least two coils should be affixed to the instrument 450 such that they are not positioned in parallel. In this way, two or more flex circuit sensors may be able to sense any magnetic flux changes in the electromagnetic field in any direction.

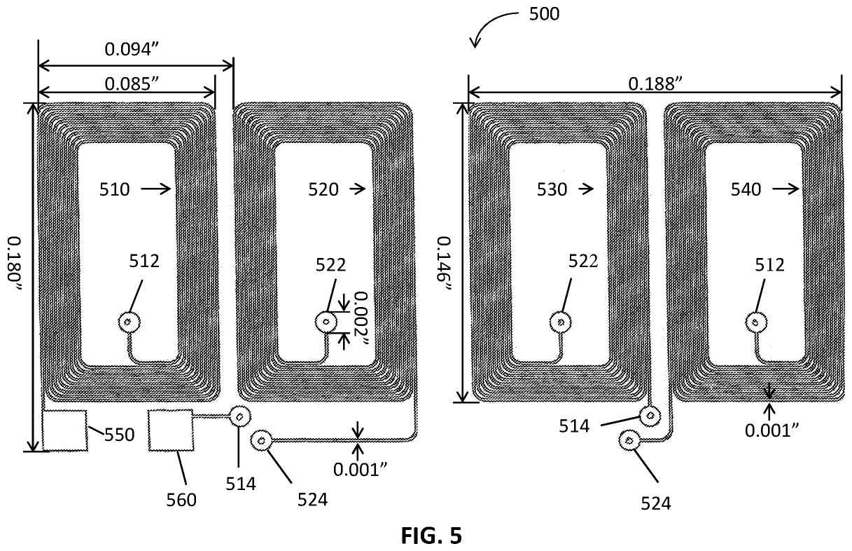

FIG. 5 shows a double layered flex circuit sensor 500 in accordance with embodiments of the present disclosure. The double layered flex circuit sensor 500 includes a first coil 510, a second coil 520, a third coil 530, and a fourth coil 540. The top layer includes the first and second coils 510 and 520 and the bottom layer includes the third and fourth coils 530 and 540. The double layered flex circuit sensor 500 further includes first and second contacts 550 and 560, and first, second, third, and fourth vias 512, 514, 522, and 524.

In one non-limiting example of the present disclosure the conductive material of each loop of any of the coils 510-540 may be approximately 9 microns thick. The thickness of the conductive material may vary based on the specifications of the flex circuit sensor 500, and can be larger or smaller than 9 microns for a particular application without departing from the scope of the present disclosure. In accordance with one embodiment of the present disclosure, each loop of the coils 510-540 of the top and bottom layers, respectively may be separated from each other by approximately 0.009 inches. The length and the width of the outermost loop of each coil may be approximately 0.146 inches and approximately 0.085 inches, respectively. The width of the conductive material may be approximately 0.001 inch. The vias may have a diameter of approximately 0.002 inches. The thickness of the flex circuit sensor 500 may be approximately 0.005 inches. The length and the width of the flex circuit sensor 500 may be approximately 0.180 and approximately 0.188 inches, respectively. The gap between closest loops of the same coil may be typically about 0.0005 inch.

As depicted in FIG. 5, the first contact 550 is connected to one end of the first coil 510 and the first via 512 is connected to the other end of the first coil 510. The first via 512 connects the first coil 510 of the top layer to one end of the fourth coil 540 of the bottom layer. The other end of the fourth coil 540 is connected to one end of the second coil 520 of the top layer through the fourth via 524. The other end of the second coil 520 is connected to one end of the third coil 530 of the bottom layer through the third via 522. The other end of the third coil 530 is connected to the contact 560 on the top layer through the second via 514. In this way, the four coils 510, 520, 530, and 540 are all connected to the first and second contacts 550 and 560, forming one sensor with the four coils connected electrically in series. Since the four coils are all connected to each other, and the number of loops in one sensor is the sum of the loops of the four coils 510, 520, 530, and 540, the result is an increase in sensitivity of the electromagnetic field.

According to a further aspect of the disclosure, the first and second coils 510 and 520 may have different rotational orientations and, likewise, the third and fourth coils 530 and 540 may have different rotational orientations. That is, if the first coil 510 has the counter clockwise orientation, the second coil 520 has the clockwise orientation. In the same way, if the third coil 530 has the counter clockwise orientation, the fourth coil 540 has the clockwise orientation. In another aspect, the first and fourth coils 510 and 540 may have the same rotational orientation and the second and third coils 520 and 530 may have the same rotational orientation.

As shown in FIG. 5, the first and second contacts 550 and 560 are made larger than the width of each loop of the coils. Generally, each coil of the flex circuit sensor 500 is coated by a non-conductive material. In an aspect, the first and second contacts 550 and 560 may not be covered by the non-conductive material so that the multi-layered flex circuit sensor 500 may be easily connected to wires which transmit the induced electrical signal (e.g., voltage and/or current) to an external apparatus, such as the tracking device 160 for incorporation into and use with the navigation and procedure software described above.

In another aspect, the first and second contacts 550 and 560 may be covered by the non-conductive material. However, the first and second contacts 550 and 560 may be in a form of a connector so that wires from an external apparatus (e.g., the tracking device 160 of FIG. 1) can be easily connected to the sensor of the flex circuit sensor 500 via the connectors. In yet another aspect, the first and second contacts 550 and 560 may have a locking mechanism that can lock a wire to connect to an external apparatus. These options may be particularly useful when applying sensors 500 to instruments in the field, where the instruments did not include such sensors from the manufacturer.

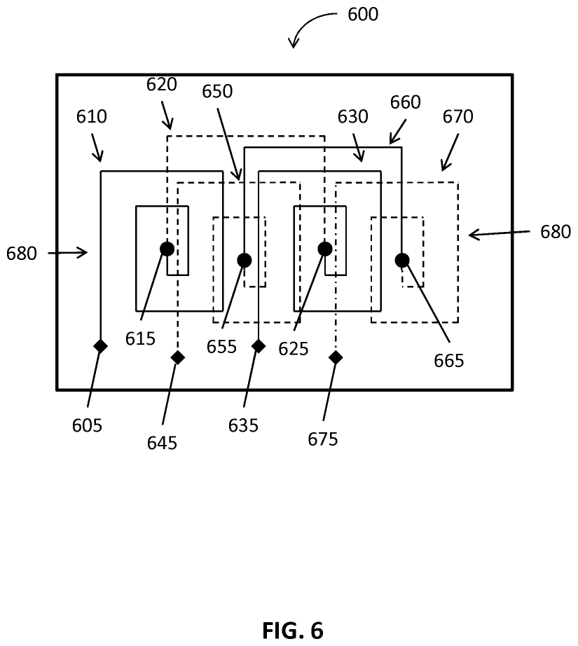

FIG. 6 shows another embodiment of a multi-layered flex circuit sensor 600. While the multi-layered flex circuit sensor 500 of FIG. 5 includes only one sensor (i.e. the four coils 510-540 electrically connected in series), the multi-layered flex circuit sensor 600 includes two sensors, each of which includes two coils on the same layer or the same side of a single layer. A first sensor 680 includes a first coil 610 and a second coil 630 on the top layer or first side and a second sensor 690 includes a third coil 650 and a fourth coil 670 on the bottom layer or second side. For convenience purpose only, in FIG. 6 loops of each coil are illustrated in a simplified schematic fashion to only a couple of loops but each loop in FIG. 6 may represent more than one loop, and the number of loops may be more in line with those of coils 510-540 of FIG. 5. The first and second coils 610 and 630 are shown in solid lines and the third and fourth coils 650 and 670 are shown in dashed lines. A first bridge 620 is located on the bottom layer and shown in dashed lines and a second bridge 660 is located on the top layer and shown in solid lines. In short, solid lines show coils and a bridge on the top layer, and dashed lines show coils and a bridge on the bottom layer.

A first contact 605 is connected to one end of the first coil 610 and a first via 615 is connected to the other end of the first coil 610. The second contact 635 is connected to one end of the second coil 630 and a second via 625 is connected to the other end of the second coil 630. The first and second coil 610 and 630 are connected by the first bridge 620 via the first and second vias 615 and 625.

A third contact 645 is connected to one end of the third coil 650 and a third via 655 is connected to the other end of the third coil 650. The fourth contact 675 is connected to one end of the fourth coil 670 and a fourth via 665 is connected to the other end of the fourth coil 670. The third and fourth coils 650 and 670 are connected by the second bridge 660 via the third and fourth vias 655 and 665.

As shown in FIG. 6, the third coil 650 is located in between the first and second vias 615 and 625 if viewed from the top layer and the second coil 630 is located in between the third and fourth vias 655 and 665 if viewed from the top layer. According to this configuration, the multi-layered flex circuit can have one sensor on each layer, or each side of a single layer without crossing conductive lines of either of the coils of the sensors. In an aspect, the first, second, third, and fourth contacts 605, 615, 635, and 675 may have a larger area than the diameter of the vias 615, 625, 655, and 665.

As depicted in FIG. 6, each coil 610, 630 on the top layer does not exactly overlap and have a matching location to the location of the third and fourth coils 650 and 670 on the bottom layer. This is in contrast to the embodiment of FIG. 5, where at least the first and fourth coils 510 and 540 overlap and the second and third coils 520 and 530 overlap. In some embodiments, all four coils of FIG. 5 overlap and have matching locations.

In an aspect, the first and second coils 610 and 630 may have a same rotational orientation (e.g., the clockwise orientation) and the third and fourth coils 650 and 670 may have a same rotational orientation (e.g., the counter clockwise orientation). In another aspect, the first and third coils 610 and 650 may have different rotational orientations.

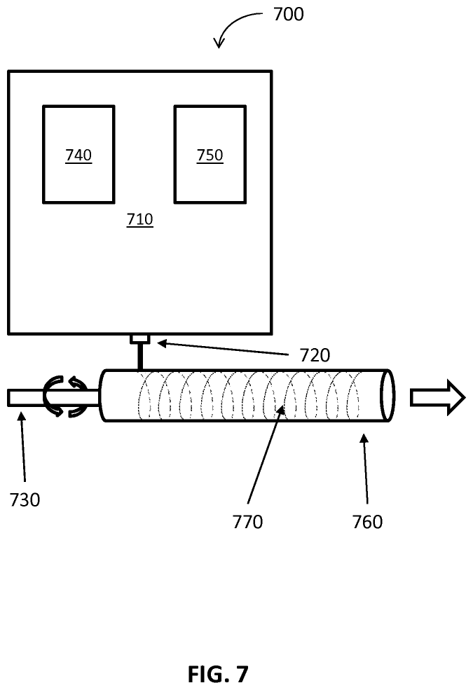

As described above, one methodology for applying sensors to instruments is via printing directly on the instruments. FIG. 7 shows a printing apparatus 700 that prints conductive and non-conductive materials directly to the desired locations of the instruments. The printing apparatus 700 includes a reservoir 710, a printing nozzle 720, and an actuating arm 730. The reservoir 710 includes a first tank 740, which contains a conductive material, and a second tank 750, which contains a non-conductive material. The printing apparatus 700 can print a circuit on any instruments 760, which can be locked into the distal end of the actuating arm 730. In an aspect, the printing apparatus may print a sensor over a polymer.

A controller of the printing apparatus 700, which is not shown in FIG. 7, controls an actuating motor, which is not shown in FIG. 7, to move the actuating arm 730. The actuating motor is fixedly connected to the proximal end of the actuating arm 730. The actuating motor can index forward and backward and rotate the actuating arm 730. In an aspect, the actuating motor may move the reservoir 710 while printing. In another aspect, the actuating motor may move the reservoir 710 and the actuating arm 730 simultaneously. For example, the actuating motor may index forward or backward the reservoir 710 while rotating the actuating arm 730. Still further, the reservoir 710 and instrument 760 may be held motionless while the printing nozzle 720, which is fluidly connected to the reservoir 710, moves about the instrument 760. Further, combinations of these techniques may be employed by those of skill in the art without departing from the scope of the present disclosure.

In one embodiment, with the proximal end of an instrument 760 locked into the distal end of the actuating arm 730, the printing nozzle 720 may start printing the conductive material contained in the first tank 740 while the actuating arm 730 is moved forward and rotated by the actuating motor. Velocities of indexing and rotating are controlled to print a helix-type sensor 770 on the instrument 760. When the velocity of indexing is faster than the velocity of rotating, the helix-type sensor 770 will have a large pitch angle or have loose loops in the helix. On the other hand, when the velocity of indexing (indexing velocity) is slower than the velocity of rotating (angular velocity), the helix-type sensor 770 will have a small pitch angle or have dense loops in the helix. Relationship between the pitch angle and velocities is shown below as follows:

$$α = {tan}^{- 1} ( \frac{{v}_{i}}{{Dv}_{θ}} ) ,$$

where α is the pitch angle, vᵢ is the indexing velocity, v_{θ} is the angular velocity of rotation in radian, and D is the cross-sectional diameter of the instrument 760. Thus, the controller may control the indexing velocity vᵢ and the angular velocity v_{θ} so that the printed circuit 770 can have a pitch angle suitable for its purpose.

In an aspect, the printing may be started from the distal end of the instrument 760 or the proximal end of the instrument 760. In a case when the printing is started from the distal end of the instrument 760, the actuating arm 730 indexes the instrument 760 forward so that the printing nozzle 720 can print the conductive material toward the proximal end of the instrument 760. In another case when the printing is started from the proximal end of the toll 760, the actuating arm 730 indexes the instrument 760 backward so that the printing nozzle 720 can print the conductive material toward the distal end of the instrument 760. In another aspect, the actuating arm 730 may change the direction of rotation so that the helix-type sensor 770 can have the counter clockwise or clockwise helix.

In an aspect, the printing nozzle 720 may print more conductive material in the beginning and end of the printing so that each end of the helix-type sensor 770 has a larger area for contact to an external apparatus.

In another aspect, after one layer of the helix-type sensor 770 is printed, the actuating arm 730 may perform a reverse indexing and rotating motion, meaning that indexing backward is performed when indexing forward is performed while the helix-type sensor 770 is printed and that counter clockwise rotation is performed when clockwise rotation is performed while the helix-type sensor 770 is printed. At the same time, the printing nozzle 720 may print the non-conductive material over the printed conductive material. In this way, the printed conductive material may be wholly covered by the non-conductive material. In another aspect, the printing nozzle 720 may be controlled to print the non-conductive material over a larger area than an area of the printed conductive material. This may give more certainty that the printed conductive material is completely covered by the non-conductive material.

After completion of printing the non-conductive material, the printing nozzle 720 may print the conductive material over the instrument 760 again. In an aspect, a new indexing velocity vᵢ′ and a new angular velocity v_{θ}′ different from the original indexing velocity vᵢ and the angular velocity v_{θ} may be selected so that new helix-type sensor may have different configuration from that of the original helix-type sensor. By repeating these steps, the instrument 760 may have several helix-type sensors.

In yet another aspect, the actuating arm 730 may control indexing forward and backward and rotation motions so that sensor may have different configurations. For example, the sensor may have a series of incomplete circles. This pattern can be obtained by rotating the actuating arm without indexing forward and by indexing forward it without rotation before completing a whole circle. The scope of the present disclosure may extend to similar or different configurations which may be readily appreciated by a person having ordinary skill in the art.

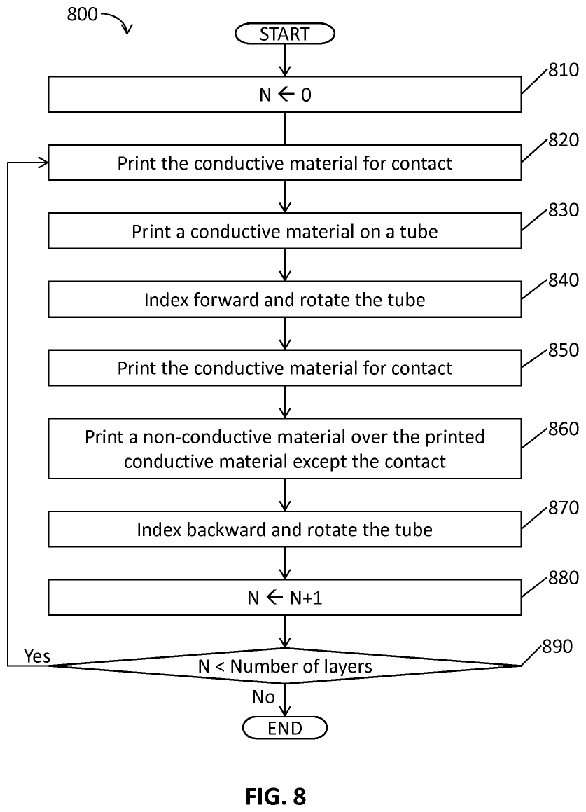

FIG. 8 shows a method 800 of printing a sensor on a surface using a printer. The sensor may be one layered or multiple layered. The method 800 starts from setting a counter N as zero in step 810. In step 820, the printer prints the conductive material for contact to an external apparatus. The contact area may be a larger than an area for printed conductive material of the sensor. In step 830, the printer prints a conductive material on the tube. While printing, in step 840, an indexing arm of the printer, which holds the tube, indexes forward or backward, and rotates the tube. Here, an indexing velocity and an angular velocity of the indexing arm may be controlled to make a specific pattern of the sensor as described above in FIG. 7.

In step 850, the printer prints the conductive material for another contact. The contacts printed in steps 810 and 850 are to be used to connect to wires which lead to and connect with an external apparatus such as the tracking device 160 of FIG. 1. The tracking device can process the sensed results to identify the location of the sensor in an electromagnetic field, as described above.

In step 860, the printer prints a non-conductive material to form a non-conductive film over the printed conductive material. While printing the non-conductive material, in step 870, the actuating arm of the printer indexes forward or backward and rotates in a direction reverse from the direction of printing the conductive material. In this way, the printed conductive material is insulated from or protected from other environments. This step concludes the printing of the sensor.

In step 880, the counter N is incremented by one. In step 890, the counter N is compared with a predetermined number of layers. If the counter N is less than the predetermined number of layers, the method 800 repeats steps 820 through 890. If the counter N is not less than the predetermined number of layers, the method is ended.

In an aspect, when the predetermined number of layers is greater than 1, a sensor printed in each layer may have different configuration, such as a helix pattern as shown in FIG. 7 and a pitch angle. In another aspect, the sensors in a multiple layers may be all connected so that the sensors only have two contacts rather than a sensor in each layer has two contacts separate from two contacts of another sensor.

Although embodiments have been described in detail with reference to the accompanying drawings for the purpose of illustration and description, it is to be understood that the inventive processes and apparatus are not to be construed as limited. It will be apparent to those of ordinary skill in the art that various modifications to the foregoing embodiments may be made without departing from the scope of the disclosure.

What is claimed is:

1. A medical instrument comprising:
a sensor having at least one coil formed of a conductive material;
a surface suitable for receiving the sensor and configured for placement in an electromagnetic field;
at least one non-conductive material covering the at least one coil of the sensor; and
at least one pair of contacts electrically connected to the at least one coil and connectable to a measurement device configured to sense an induced electrical signal based on a magnetic flux change of the electromagnetic field,
wherein a location of the medical instrument in a coordinate system of the electromagnetic field is identified based on the induced electrical signal in the sensor.
